(19) United States
(12) Patent Application Publication
(10) Pub. No.: US 2014/0276564 A1
(43) Pub. Date: Sep. 18, 2014

(54) PUMP CONTROLLER AND PUMP FOR INDIVIDUALIZED HEALTHCARE USE

(71) Applicants: BAXTER INTERNATIONAL INC., Deerfield, IL (US); BAXTER HEALTHCARE SA, Glattpark (Opifkon) (CH)

(72) Inventor: Dennis I. Schneider, Nashua, NH (US)

(73) Assignees: BAXTER HEALTHCARE SA, Glattpark (Opifkon) (CH); BAXTER INTERNATIONAL INC., Deerfield, IL (US)

(21) Appl. No.: 13/830,958

(22) Filed: Mar. 14, 2013

(51) Int. Cl. A61M 5/172

(52) U.S. Cl. CPC A61M 5/1723
USPC 604/506; 604/151

(57) ABSTRACT

According to an aspect of the present disclosure, a pump system for individualized healthcare use includes an infusion pump, a display, an input interface, and a pump controller. The pump controller is associated with a memory in which a closed set of predefined infusion therapies and a personalized identifier associated with an individual patient are stored. The pump controller is programmed to display the personalized identifier; to operate according to a first operational state, wherein the pump controller receives an input via the input interface, selects a predefined infusion therapy according to the input received, and controls the infusion pump to provide the selected predefined infusion therapy; to operate according to a second operational state; and to receive an operational state code via the input interface, and to change from the first to the second operational state only when the operational state code is received.

### BACKGROUND

The present disclosure relates to a pump controller and an associated infusion pump for healthcare use, and in particular a pump controller and an associated infusion pump for individualized healthcare use.

Intravenous (IV) fluid administration is commonplace within healthcare facilities, such as hospitals and nursing homes. For example, drugs may be administered to the patient intravenously. Alternatively, electrolytes may be intravenously administered to the patient. As a still further example, the patient's nutritional needs may be met, in whole or in part, through parenteral nutrition therapy.

IV therapy typically involves the use of an infusion pump (such as a peristaltic pump) in combination with a pump controller to provide the fluid from a container at a desired rate. The combination of the pump controller and the pump may be referred to as a “pump,” even though the system includes both the infusion pump or pump channel and the pump controller. The infusion pump may be integral with the pump controller, in that the infusion pump and the pump controller are defined as a single unit (for example, by being disposed in a common housing). Alternatively, the infusion pump and the pump controller may be attachable to and detachable from each other.

A healthcare facility may own and operate hundreds of pumps, which pumps may be used in the treatment of hundreds, if not thousands, of patients each year. As such, these pumps must be able to provide a wide range of therapies. To provide the necessary functional flexibility, current pump controllers have become sophisticated programmable computerized devices. Of course, it is also true that the programming of the therapy parameters into these sophisticated pump controllers is performed by skilled healthcare professionals in the settings described above. The sophistication of the user is taken into consideration in the design of these pumps.

It will also be recognized that there is a growing market for home healthcare services. Moreover, the nature of healthcare services being provided in the home healthcare setting, as opposed to at a healthcare facility such as a hospital, is increasing in sophistication. For example, it is not uncommon for a doctor to prescribe intravenous therapies to be carried out in the home healthcare setting. In such a case, the prescription may include certain fluids (drugs in solution, electrolyte solutions, etc.) to be administered intravenously, as well as the pump used to deliver those fluids.

Given the sophistication of the pumps and the relative unsophistication of the patient, such a therapy will typically require a healthcare professional, such as a visiting nurse, to program the pump and oversee the therapy. The involvement of a healthcare professional obviously increases the cost of the therapy and eliminates some of the independence that the patient would otherwise enjoy in the home healthcare setting. Alternatively, if the patient attempts to program the pump him or herself, the patient may be unable to complete the programming and/or become frustrated with the process.

As set forth in more detail below, the present disclosure sets forth a system embodying advantageous alternatives to the conventional devices and approaches discussed above.

### SUMMARY

According to an aspect of the present disclosure, a pump system for individualized healthcare use includes an infusion pump, a display, an input interface, and a pump controller coupled to the infusion pump, the display and the input interface. The pump controller is associated with a memory in which a closed set of predefined infusion therapies and a personalized identifier associated with an individual patient are stored. The pump controller is programmed to display the personalized identifier on the display; to operate according to a first operational state, wherein the pump controller receives an input via the input interface, selects a predefined infusion therapy from the closed set according to the input received, and controls the infusion pump to provide the selected predefined infusion therapy; to operate according to a second operational state, wherein the pump controller does not operate according to the first operational state; and to receive an operational state code via the input interface, and to change from the first operational state to the second operational state only when the operational state code is received.

According to another aspect of the present disclosure, a method of operating a pump system, wherein the pump system includes an infusion pump and a display, for individualized healthcare use includes displaying a personalized identifier on the display, the personalized identifier associated with an individual patient. The method also includes receiving an input; operating the infusion pump, in a first operational state, according to a predefined infusion therapy selected from a closed set of predefined infusion therapies associated with the individual patient according to the input; receiving an operational state code; and operating the infusion pump, in a second operational state, different than the first operational state when the operational state code is received.

According to a further aspect of the present disclosure, a method of providing individualized healthcare includes storing a closed set of predefined infusion therapies and a personalized identifier associated with an individual patient in a memory associated with a pump controller of a pump system. The pump system includes an infusion pump, a display, an input interface, and the pump controller. The pump controller is coupled to the infusion pump, the display and the input interface. The pump controller is programmed to display the personalized identifier on the display; to operate according to a first operational state, wherein the pump controller receives an input via the input interface, selects a predefined infusion therapy from the closed set according to the input received, and controls the infusion pump to provide the selected predefined infusion therapy; to operate according to a second operational state, wherein the pump controller does not operate according to the first operational state; and to receive an operational state code via the input interface, and to change from the first operational state to the second operational state only when the operational state code is received. The method also includes providing a master code to the pump controller, the pump controller operating according to the first operational state and the second operational state when the code is received and preventing operation according to the first operational state or the second operational state when the master code is not received; and shipping the pump system with one or more containers each containing a fluid associated with one or more of the closed set of predefined infusion therapies.

Additional aspects of the disclosure are defined by the claims of this patent.

### BRIEF DESCRIPTION OF THE FIGURES

It is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 is a schematic view of a pump system according to the present disclosure;

FIG. 2 is a simulated screenshot of a display of a personalized identifier according to the present disclosure;

FIG. 3 is a simulated screenshot of a display of a request for an input associated with an infusion therapy selection;

FIG. 4 is a simulated screenshot of a display of a prompt for an operational state code;

FIG. 5 is a simulated screenshot of a display of a prompt for a master code; and

FIG. 6 is a flowchart of a method of operating the pump system of FIG. 1 according to an embodiment of the present disclosure.

### DETAILED DESCRIPTION

An embodiment of the present disclosure of a pump system 100 for individualized healthcare use is illustrated in FIG. 1. The pump system 100 includes an infusion pump 102, a display 104, at least one input interface 106, and a pump controller 108. The pump controller 108 is coupled to the infusion pump 102, the display 104 and the input interface 106 as illustrated.

The display 104 may be a liquid crystal display (LCD), a cathode ray tube (CRT) display, a light emitting diode (LED) display, etc. It may be advantageous for the display 104 to be a touchscreen, in which case the display 104 may also function as the at least one input interface 106. However, the at least one input interface 106 may be defined by equipment separate and apart from the display 104, such as push buttons, a keyboard, a keypad, or a pointing device (e.g., mouse), and may also include an optical scanning device (e.g., a barcode scanner), a magnetic card reader, a solid-state memory reader (e.g., a flash memory reader), an optical drive (e.g., CD-ROM, DVD-ROM), a floppy drive, a radio-frequency identification reader (RFID), mobile phone, tablet computer, personal computer, or any other known input device.

The pump controller 108 includes at least one processor 110 (e.g., a microprocessor) and at least one memory 112. The memory 112 includes instructions, in the form of one or more programs, routines, modules, etc., that are executed by the processor 110, and when executed by the processor 110 cause the pump system controller 108 to carry out the functions associated with the operation of the pump system 100. In at least this regard, the pump controller 108 may be referred to as being programmed to carry out one or more activities. The memory 112 may be implemented as semiconductor memories, magnetically readable memories, and/or optically readable memories, for example. The memory 112 may be in form of read-only memory (ROM) and random access memory (RAM). By way of example and not limitation, the ROM may take many different forms, including erasable programmable ROM (EPROM) and electrically erasable programmable ROM (EEPROM).

The memory 112 may also have stored there a closed set of predefined infusion therapies associated with an individual patient. The infusion therapies are predefined in that the parameters required by the pump controller 108 to control the infusion pump 102 to provide the therapy are stored in the memory prior to the system 100 providing the therapy, rather than being received via the input interface 106 at the time the system 100 provides the therapy. According to certain embodiments, the predefined infusion therapies are stored prior to the pump system being provided to a patient for use. The set may include one or more such predefined infusion therapies. The set is closed in that the set is not capable of being changed by using the input interface 106 only; for example, as explained in greater detail below, an operational state code must be used in conjunction with the input interface 106 to reprogram the set of predefined infusion therapies.

It will be recognized that the pump system 100 may also include other elements as well. For example, the pump system 100 may include an additional (or second) input interface 120, which interface may permit communication with the pump controller 108 in a manner the same as or different than the input interface 106. The input interface 120 may also include equipment capable of communicating via a wired connection (e.g., Ethernet, USB, Firewire, Lighting, etc.) or a wireless connection (e.g., an IEEE 802.11 standard, ZigBee, Bluetooth®, etc.). The pump system 100 may also include other output interfaces 122 for communicating with a user or the patient; an audio speaker would be one example of an output interface 122.

According to the present disclosure, the pump controller 108 may be programmed to carry out a number of actions, and to operate in one or more operational states. For example, the pump controller 108 may be programmed to display a personalized identifier 130 associated with the patient on the display 104 as illustrated in FIG. 2. As illustrated, the personalized identifier 130 may be in the form of an alphanumeric string, such as a name (e.g., first name, last name). Of course, the personalized identifier 130 may take other forms, as may be helpful when the patient has one or more pumps in a single dwelling. In such a circumstance, the identifier may be associated with the patient, as well as with a particular room in the dwelling (e.g., “Mike's Living Room Pump”) or with a particular therapy provided by the pump (e.g., “Mike's Nutritional Pump”). The personalized identifier 130 may be displayed only at start-up of the pump system 100, or may be continuously displayed in one area or region of the display 104 even as other information is displayed in other areas or regions of the display 104. This action may be optional according to certain embodiments of the present disclosure.

The pump controller 108 may also be programmed to operate according to a first operational state. In this first operational state, the pump controller 108 may receive an input via the input interface 106, select a predefined infusion therapy from the closed set according to the input received, and control the infusion pump 102 to provide the selected predefined infusion therapy. As illustrated in FIG. 3, during this operational state, the pump controller 108 may also control the display 104 to provide a prompt to the user to provide an input via the input interface 106. For example, as illustrated, the pump controller 108 may control the display 104 to display textual and/or graphical information to indicate to the user which predefined infusion therapy will be selected by the pump controller 108 in response to the user manipulation of the input interface 106. For example, where the input interface 106 is a keyboard, the display 104 may include prompts 132, 134, 136 to indicate that if the left, center or right keys are touched or depressed, a first, second or third therapy will be selected, respectively. The prompts 132, 134, 136 may include information such as the drug for example, so as to provide additional guidance to the user. While three prompts 132, 134, 136 are illustrated, it is understood that rather than three prompts 132, 134, 136 being displayed, a single prompt 132 may be displayed. As a further alternative, additional prompts (i.e., more than three) may be displayed.

As illustrated, the prompts 132, 134, and 136 correspond to a total parental nutrition (TPN) therapy, a Vancomycin therapy and a hydration therapy. Thus, it will be recognized that the prompts may identify a particular drug, biologic, etc. being administered (e.g., Vancomycin), or may refer to the general type of therapy being provided (e.g., TPN, hydration). It will also be recognized that the prompts may include additional information regarding the therapy, which additional information may assist in orienting the user to connect the pump system 100 to the appropriate container(s) 150 that contain the relevant fluids. For example, the prompt could information regarding the total volume to be delivered, which information could also be displayed on the container 150. Of course, it is also possible that the container 150 may be marked to correspond to whatever graphical and/or textual identifiers are display in the prompts 132, 134, 136.

In fact, according to certain embodiments of the present disclosure, the pump controller 108 will only receive inputs from the interface 106, select predefined infusion therapies from the closed set according to those inputs, and control the pump 102 to provide the selected therapies in the first operational state. In this fashion, the user is not able to vary the parameters of the therapy (e.g., drug, concentration, volume, rate, time) or add/delete therapy options. Even a very sophisticated pump controller that is capable of a wide range of additional functionality may be programmed to provide such a first operational state. The programming of the pump controller 108 to provide this functionality may involve setting off a particular section of the memory 112 for use in storing the predefined infusion therapies, or may involve rewriting existing databases so that the amount of information contained in the database only covers the therapies intended to be provided by the pump system 100. For example, in those pump systems 100 that include an on-board drug library, used to check if the parameters for a particular therapy exceed safe limits for example, the drug library may be rewritten such that only the drugs, concentrations, rates and volumes to be provided by the pump system 100 are selectable.

In regard to the parameters that may be programmed for a specific therapy, this may include one or more of the following (which are provided by way of an example only, and not by way of limitation): a dose mode, an identification of the drug or fluid, a concentration of the drug or fluid, a dose for the drug or fluid, a rate (or rates) at which the fluid is administered, a volume of the fluid to be administered, and a period of time (or time periods) over which the fluid will be administered. As to the dose mode, this may refer to a continuous infusion (where the therapy continues at a first rate until no longer required, or at a first rate for a predefined period of time or until a predefined volume has been infused), cyclic (where the drug or fluid therapy ramps up at a first volume rate (e.g., mL/hr) to a second volume rate at which is therapy is continued for a prescribed period of time, after which the therapy tapers down at a third volume rate (which may be the same as the first rate) until the total infusion time has elapsed) or a multi-step program (where an infusion therapy is administered in a series of steps or stages, with each step or stage potentially involving infusion at a different rate and/or volume, and/or for a different period (or amount) of time). The parameter information described above may be required so that the pump system 100 may carry out the dose mode selected (i.e., the dose mode may require information such as rate, volume and time). Alternatively, the parameter information may be required as part of an initial error check, performed by the pump system 100 upon receipt of any infusion therapy, to limit the chances that the infusion therapy could cause adverse drug events, which check may still be performed even when the system 100 receives the parameter information as part of a predefined infusion therapy.

While it may be desirable to operate the pump system 100 only in the first operational state, it may also be desirable to provide a second (or third, etc.) operational state wherein the pump controller 108 provides functionalities not available or possible in the first operational state. For example, in the second operational state, the user may be permitted to alter the closed set of predefined infusion therapies. Alternatively, the second operational state may permit the user to cause the pump controller 108 to carry out on-board diagnostic programs, which may be useful in determining why the pump controller 108 and/or pump 102 are not operating as expected. Still other alternatives are possible as well, such as a second operational state used to modify the closed set of predefined infusion therapies and a third troubleshooting state. As another example, a second operational state might permit only view of the pump's recent event logs, a third operational state might allow overriding the operating parameters for the current dose, and a fourth operational state might allow reprogramming of the internal does delivery program (e.g., change rate). In any event, the pump controller 108 is programmed to operate according to such a second (or additional) operational state so that the controller 108 does not operate according to the first operational state.

Because it is desired to have the pump system 100 operate in the first operational state under most circumstances, a code may be required to shift between the first and second (or additional) operational states or to change from the first operational state to the second (or additional) operational state. To this end, the pump controller 108 may be programmed to receive an operational state code via the input interface 106, and to change from the first operational state to the second operational state only when the operational state code is received. For example, the pump controller 108 may be directed to enter the second operational state by a technician to access the additional functionality of the pump system 100 (and in particular the pump controller 108) or by the user as part of a telephonic troubleshooting session. When the second operational state is used to provide for local (i.e., in the same vicinity or dwelling) or remote (i.e., via long-distance communication, such as a telephone or computer network) analysis and (attempted) correction of the operation of the pump system 100, the second operational state may also be referred to as a troubleshooting state. As noted above, during this troubleshooting state, the pump controller 108 (and associated memory 112) may be reprogrammed.

As noted above, the pump system may have multiple operational states, each associated with different activities. For example, a second operational state might permit only view of the pump's recent event logs, a third operational state might allow overriding the operating parameters for the current dose, and a fourth operational state might allow reprogramming of the internal does delivery program (e.g., change rate), as mentioned above. Each operational state may be associated with a different operational code, which code may be required to shift between the first and second, third or fourth operational states or to change from the first operational state to the second, third or fourth operational state. As a consequent, a considerable degree of flexibility may be maintained in a modular or compartmentalized fashion relative to the user, while presenting access primarily to the first operational state.

FIG. 4 illustrates a screen that may be displayed on the display 104 as directed by the pump controller 108. The screen includes a prompt 138 to instruct the user to contact patient services for further assistance, and to enter the second operational code (“troubleshooting code”) using the input interface 106 when instructed. As such, in addition to the programming described above, the pump controller 108 may also be programmed to request an operational state code from the user in conjunction with receiving the code and to change from the first operational state to the second operational state when the operational state code is received.

It may also be desirable to program the pump controller 108 in such a way that the pump controller 108 will only operate when used by the patient whose therapies are stored in the memory 112. For example, it would be desirable to prevent other parties from operating the pump system 100, other than the intended user. In particular, if would be desirable to prevent other parties from obtaining the pump system 100 without permission of the pump system owner and attempting to sell the pump system 100 to third parties. This is a concern of particular interest to the home healthcare market, in that most healthcare facilities have inventory and security measures that prevent their pump systems from being removed from the premises. When placed in a home healthcare setting, it may not be feasible for the party that owns the pump system 100 (and which rents or leases the pump system 100 to the patient) to control the movement of the pump system 100.

Consequently, it may also be desirable that the pump controller 108 is programmed to receive a master code via the input interface 106, to operate according to the first operational state or the second operational state when the master code is received, and to prevent operation according to the first operational state or the second operational state when the master code is not received. In this fashion, the use of the master code is a prerequisite to use of the pump system 100. If the master code is not entered, the pump system 100 may take action to deny the use of the pump 102, completely cease to operate or refuse to operate and instruct the user to contact the support provider.

In fact, the pump controller 108 may be programmed to request the master code, as illustrated in FIG. 5, via a prompt 140. In fact, to ensure greater security, the pump controller 108 may be programmed to accept a given master code for a limited period of time, after which a new master code must be received by the pump controller 108 to permit operation of the pump system 100. For example, the pump controller 108 may be programmed to periodically request the master code.

According to certain embodiments of the present disclosure, in the first operational state, the pump controller 108 may only either (i) receive an input via the input interface 106, (ii) select a predefined infusion therapy from the closed set, and (iii) control the infusion pump 102 to provide the selected predefined infusion therapy or receive an operational or master code via the input interface 106. According to such an embodiment, irrespective of the sophistication of the pump system 100, the patient is presented with simplified version of the pump system 100 for daily use. Under exceptional circumstances, the operational code may be used to access the more sophisticated functionality of the pump system 100, and thereby resolve issues either locally or remotely that would not be possible to do with a pump that was designed to carry out only a single predefined infusion therapy.

As mentioned above, according to certain embodiments of the present disclosure, the pump system 100 may include a second input interface 120, and the pump controller 108 may be coupled to the second interface 120. According to such an embodiment, the pump controller 108 may receive the closed set of predefined infusion therapies and the personalized identifier via the second interface 120. For example, the second interface 120 may be a wireless interface. According to such an embodiment, it may not be necessary to use the first interface 106 to provide the closed set of predefined infusion therapies and the personalized identifier to the pump controller 106, although the use of the first interface may remain as an optional communication path.

It will also be recognized that the pump system 100 may include one or more containers 150. Each container 150 may contain one or more fluid(s) associated with at least one of the predefined infusion therapies. As illustrated, the pump system 100 may be used in the first operational state with one container 150 in the form of a flexible bag connected to the patient via an administration set (and associated equipment) 152. In fact, the pump system 100 may be provided to the patient with a plurality of containers (and associated equipment, such as administration sets) for use over a longer period of time than a single treatment (e.g., a week, a month, etc.).

Having thus described the structure of the system 100 with reference to FIGS. 1-5, the operation of the system is described with reference to FIG. 6. The method 200 of operating a pump system for individualized healthcare use (such as the pump system 100 including the infusion pump 102 and a display 104) may optionally start at block 202 with the pump controller 108 controlling the display 104 to display a prompt to request the master code (see FIG. 5). If the master code is received at block 204, then the method 200 proceeds to block 206 (i.e., the pump system 100 may operated in either the first operational state or the second operational state). If the master code is not received at block 204, the pump controller 108 prevents operation of the infusion pump 102 according to either the first operational state or the second operational state. For example, the method 200 may return to block 202.

At block 206, the pump controller 108 may control the display 104 to display a personalized identifier on the display 104, the personalized identifier associated with an individual patient. See FIG. 2. The method 200 may then proceed to block 208, and the pump controller 108 may control the display 104 to request an input via the input interface 106 relative to the desired infusion therapy. See FIG. 3. The method 200 passes to block 210, wherein it is determined if an input is received. If an input is received, the method may proceed to block 212.

At block 212, the pump controller 108 operates the infusion pump 102, in a first operational state, according to a predefined infusion therapy selected from the closed set of predefined infusion therapies associated with the individual patient according to the input received at block 210. The method 200 may check at block 214 if there is a reason to exit the first operational state and proceed to a second operational state. For example, the method 200 may leave the first operational state of block 212 if the pump 102 provides a malfunction input to the pump controller 108. In such a case, the method may proceed from blocks 212, 214 to block 216.

At block 216, the pump controller 108 may control the display 104 to display a prompt to request an operational state (in this case, troubleshooting) code. See FIG. 4. The method 200 may remain at blocks 216, 218 until the operational state code is received. Once the operational state code 218 is received, the pump controller 108 may operate, in a second operational state, different than the first operational state at block 220. For example, the pump controller 108 may prevent the infusion pump 102 from operating according to the selected predefined infusion therapy parameters, and permit access to on-board diagnostics. Once the user has completed the troubleshooting at block 220, the method 200 may return to block 212. It will be readily recognized how the steps of blocks 216, 218, 220 may be adapted (e.g., repeated) to address additional operational states and associated operational codes.

The method 200 may optionally return to block 202 to make an additional request for the master code after a period of time has elapsed, as indicated at block 222. The period of time may reoccur, such that the method returns to block 202 periodically. Alternatively, the period of time may be measured (determined) once and only once according to a predetermined amount (e.g., one month). As a further alternative, the period of time may be measured once and only once according to a variable amount (i.e., for some random time period).

Having thus described the structure and operation of the pump system 100 according to the present disclosure, the method of providing individualized healthcare now may be discussed relative to the pump system 100. According to such a method, a first party captures information regarding a patient and a set of infusion therapies prescribed for the patient. The first party may be, for example, a healthcare product provider such as may fill prescriptions prepared by a healthcare professional (e.g., doctor, physician's assistant, etc.). The information regarding the patient may include a personalized identifier (e.g., name), and the information regarding the therapies may include the drug, its concentration, the volume to be infused, rate or rate limits, etc. The method of capture may be manual or automated (e.g., from an electronic facsimile or scan of a paper prescription, from an electronic medication order, etc.).

The healthcare product provider then stores a closed set of predefined infusion therapies and a personalized identifier associated with an individual patient in the memory 112 associated with the pump controller 108 of the pump system 100. The closed set of predefined therapies and the personalized identifier may be referred to as a personality associated with the individual patient, which personality may also include the codes, prompts (e.g., prompts 130, 132, 134, 136, 138, 140), messages, etc. As such, the prompts 130, 132, 134, 136, 138, 140 that may be stored for the personality designed for one patient may differ relative to those elements designed for another patient, in the same way that the therapies to be administered to one patient may differ from the therapies administered to another patient. The pump system 100 may include the infusion pump 102, the display 104, the input interface 106, and the pump controller 108, as described above.

For example, the pump controller 108 may be coupled to the infusion pump 102, the display 104 and the input interface 106, and the pump controller 108 may be programmed to display the personalized identifier on the display 104; to operate according to a first operational state, wherein the pump controller 108 receives an input via the input interface 106, selects a predefined infusion therapy from the closed set according to the input 106 received, and controls the infusion pump 102 to provide the selected predefined infusion therapy; to operate according to a second operational state, wherein the pump controller 108 does not operate according to the first operational state; and to receive an operational state code via the input interface 106, and to change from the first operational state to the second operational state only when the operational state code is received.

To enable the pump system, the healthcare product provider may provide a master code to the pump controller 108, the pump controller 108 operating according to the first operational state and the second operational state when the code is received and preventing operation according to the first operational state or the second operational state when the master code is not received. Alternatively, the master code may be provided by the user (e.g., the patient). Finally, the healthcare product provider may ship the pump system 100 with one or more containers 150 each containing a fluid associated with one or more of the closed set of predefined infusion therapies.

When the patient has completed his or her use of the pump system 100, the healthcare product provider may receive the pump system 100, and erase the closed set and the personalized identifier associated with the individual patient. The healthcare product provider may then store a closed set of predefined infusion therapies and a personalized identifier associated with another individual patient in the memory associated with the pump controller, provide the pump system 100 with the providing the master code, and ship the pump system 100 with one or more containers 150 each containing a fluid associated with one or more of the closed set of predefined infusion therapies associated with the another individual patient.

To facilitate the process of storing the closed set and the personalized identifier comprises, the healthcare product provider may transmit the closed set and the personalized identifier wirelessly to the pump controller 108 of the pump system 100, via the second input interface 120 for example. The transmission of the closed set and the personalized identifier via the second input interface 120 to the pump controller 108 of the pump system 100 may also occur via a wired or any other means of communication. Alternatively, the healthcare product provider may input the closed set and the personalized identifier using the first input interface 106.

It is believed that the pump system according to the present disclosure may provide one or more advantages, one or more of which may be provided in a particular embodiment of the present disclosure. The pump system described herein may permit a sophisticated pump system to be provided for individualized healthcare use, for example by a patient in the home healthcare setting, with decreased concern that the sophistication of the pump system will discourage the patient from using the system. At the same time, the pump system may maintain its sophisticated functionality, effectively screened from the patient, so that if the additional functionality is required, for example to diagnosis a pump error, it is available for use. Moreover, the pump system may provide additional safeguards against the authorized use and sale of the pump system. Furthermore, display of personalized identifiers may facilitate recognition of the pump as associated with the patient, simplifying verification of the association.

Although the preceding and following text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

It should be understood other changes and modifications to the presently preferred embodiments described herein would also be apparent to those skilled in the art. Such changes and modifications can be made without departing from the spirit and scope of the present subject matter and without diminishing its intended advantages. It is therefore intended that such changes and modifications be covered by the appended claims.

What is claimed is:

1. A pump system for individualized healthcare use comprises:
an infusion pump;
a display;
an input interface; and
a pump controller coupled to the infusion pump, the display and the input interface, the pump controller associated with a memory in which a closed set of predefined infusion therapies and a personalized identifier associated with an individual patient are stored and the pump controller programmed:
to display the personalized identifier on the display;
to operate according to a first operational state, wherein the pump controller receives an input via the input interface, selects a predefined infusion therapy from the closed set according to the input received, and controls the infusion pump to provide the selected predefined infusion therapy;
to operate according to a second operational state, wherein the pump controller does not operate according to the first operational state; and
to receive an operational state code via the input interface, and to change from the first operational state to the second operational state only when the operational state code is received.

2. The pump system according to claim 1, wherein the predefined infusion therapy includes a dose.

3. The pump system according to claim 1, wherein the second operational state comprises a troubleshooting state wherein the pump controller may be reprogrammed.

4. The pump system according to claim 1, wherein the pump controller is programmed to receive a master code via the input interface, to operate according to the first operational state or the second operational state when the master code is received, and to prevent operation according to the first operational state or the second operational state when the master code is not received.

5. The pump system according to claim 4, wherein the pump controller is programmed to request the master code.

6. The pump system according to claim 5, wherein the pump controller is programmed to periodically request the master code.

7. The pump system according to claim 1, wherein in the first operational state, the pump controller only either (i) receives an input via the input interface, (ii) selects a predefined infusion therapy from the closed set, and (iii) controls the infusion pump to provide the selected predefined infusion therapy or receives an operational or master code via the input interface.

8. The pump system according to claim 1, further comprising a second interface, wherein the pump controller is coupled to the second interface and the closed set of predefined infusion therapies and the personalized identifier are received via the second interface.

9. The pump system according to claim 8, wherein the second interface is a wireless interface.

10. The pump system according to claim 1, wherein the closed set of predefined infusion therapies and the personalized identifier are received via the first interface.

11. The pump system according to claim 1, further comprising one or more containers each containing a fluid associated with at least one of the predefined infusion therapies.

12. A method of operating a pump system for individualized healthcare use, the pump system including an infusion pump and a display, the method comprising:
displaying a personalized identifier on the display, the personalized identifier associated with an individual patient;
receiving an input;
operating the infusion pump, in a first operational state, according to a predefined infusion therapy selected from a closed set of predefined infusion therapies associated with the individual patient according to the input;
receiving an operational state code; and
operating the infusion pump, in a second operational state, different than the first operational state when the operational state code is received.

13. The method of operating a pump system according to claim 12, wherein the second operational state is a troubleshooting state.

14. The method of operating a pump system according to claim 12, further comprising:
requesting a master code;
operating the infusion pump according to either the first operational state or the second operational state when the master code is received; and
preventing operation of the infusion pump according to either the first operational state or the second operational state when the master code is not received.

15. The method of operating a pump system according to claim 14, further comprising periodically requesting the master code.

16. The method of operating a pump system according to claim 12, further comprising receiving the closed set of predefined infusion therapies via a wireless communication.

17. A method of providing individualized healthcare, the method comprising:
storing a closed set of predefined infusion therapies and a personalized identifier associated with an individual patient in a memory associated with a pump controller of a pump system, the pump system comprising an infusion pump, a display, an input interface, and the pump controller,
the pump controller coupled to the infusion pump, the display and the input interface, and the pump controller programmed to display the personalized identifier on the display; to operate according to a first operational state, wherein the pump controller receives an input via the input interface, selects a predefined infusion therapy from the closed set according to the input received, and controls the infusion pump to provide the selected predefined infusion therapy; to operate according to a second operational state, wherein the pump controller does not operate according to the first operational state; and to receive an operational state code via the input interface, and to change from the first operational state to the second operational state only when the operational state code is received;
providing a master code to the pump controller, the pump controller operating according to the first operational state and the second operational state when the code is received and preventing operation according to the first operational state or the second operational state when the master code is not received; and
shipping the pump system with one or more containers each containing a fluid associated with one or more of the closed set of predefined infusion therapies.

18. The method of providing individualized healthcare according to claim 17, further comprising:
receiving the pump system;
erasing the closed set and the personalized identifier associated with the individual patient;
storing a closed set of predefined infusion therapies and a personalized identifier associated with another individual patient in the memory associated with the pump controller;
providing the master code;
shipping the pump system with one or more containers each containing a fluid associated with one or more of the closed set of predefined infusion therapies associated with the another individual patient.

19. The method of providing individualized healthcare according to claim 17, wherein storing the closed set and the personalized identifier comprises transmitting wirelessly the closed set and the personalized identifier.
